(19) United States
(12) Patent Application Publication
(10) Pub. No.: US 2007/0208319 A1
(43) Pub. Date: Sep. 6, 2007

(54) Flat type disposable diaper

(75) Inventor: Masanori Minato, Haga-gun (JP)

Correspondence Address: BIRCH STEWART KOLASCH & BIRCH, PO BOX 747, FALLS CHURCH, VA, 22040-0747, US

(73) Assignee: Kao Corporation, Tokyo (JP)

(21) Appl. No.: 11/713,680

(22) Filed: Mar. 5, 2007

(30) Foreign Application Priority Data
Mar. 6, 2006 (JP) 2006-59031

(51) Int. Cl. A61F 13/15

(52) U.S. Cl. 604/385.26; 604/385.28

(57) ABSTRACT

A flat type disposable diaper 1 having a liquid permeable topsheet 2, a liquid impermeable or repellent backsheet 3, a liquid retentive absorbent member 4 interposed between the two sheets, a pair of anti-leakage cuffs 6 having standing gather-forming elastic members 61, and a pair of leg flaps 7 having leg gather-formfing elastic members 71. The diaper 1 is sectioned into a front section A, a crotch section B, and a rear section C along the longitudinal direction. The absorbent member 4 includes a higher weight region 4A and a lower weight region 4B. The lower weight region 4B is located in the rear section C, and the higher weight region 4A is located in the crotch section B. At least one of the standing gather and the leg gather is substantially absent in the rear section C having the lower weight region 4B.

### FIELD OF THE INVENTION

The present invention relates to a flat type disposable diaper.

### BACKGROUND OF THE INVENTION

Today, optimization of materials constituting disposable diapers has been promoted to increase the absorbency. Thickness reduction of an absorbent member (or core) of disposable diapers has also been studied in order to improve wearing comfort, increase packaging efficiency and reduce the material cost To achieve the thickness reduction while maintaining capability of capturing a large amount of urine, diapers are often designed to have increased absorbency in a region from the longitudinally middle portion forward (toward the front waist portion).

For example, a disposable diaper having an absorbent member with its weight per unit area varied regionally in the longitudinal direction is proposed (see U.S. Pat. No. 4,685,915A and US 2003236510A1).

U.S. Pat. No. 4,685,915A discloses a flat type disposable diaper including a topsheet, a backsheet, and an absorbent member intermediate between the two sheets, having a substantially oblong shape, having a pair of leg flaps elasticized with a gather-forming elastic member extending along both sides of the diaper. The diaper is featured by its absorbent member having a lower weight per unit area in the rear section of the diaper than in the longitudinally middle section and the front section of the diaper.

US 2003236510A1, which is commonly assigned to the same assignee of the present invention, proposes a flat type disposable diaper having a liquid permeable topsheet, a liquid impermeable backsheet, and a liquid retentive absorbent member intermediate between the two sheets, and having a substantially oblong shape, the absorbent member containing a superabsorbent polymer and pulp fiber. The absorbent member has a higher weight region having more superabsorbent polymer and more pulp fiber than the rest. The higher weight region is located at a position nearer to the longitudinal end of the front section than to that of the rear section. The higher weight region and the rest of the absorbent member constitute a unitary structure with physical continuity.

### SUMMARY OF THE INVENTION

The present invention provides a flat type disposable diaper having a topsheet, a backsheet, an absorbent member interposed between the topsheet and the backsheet, a pair of anti-leakage cuffs elasticized with a standing gather-fonning elastic member, and a pair of leg flaps elasticized with a leg gather-forming elastic member. The diaper has a longitudinal direction and is sectioned into a front section, a crotch section, and a rear section. The absorbent member includes a higher weight region and a lower weight region. The lower weight region is located in the rear section, and the higher weight region in the crotch section. At least one of the standing gather and the leg gather is substantially absent in the rear section where the lower weight region of the absorbent member is provided.

### BRIEF DESCRIPTION OF THE DRAWING

FIG. 1 is a perspective of a flat type disposable diaper incorporating a first embodiment of the present invention in its relaxed state.

FIG. 2 is a plan of the diaper of FIG. 1 in its flat-out state with every elastic member stretched out flat.

FIG. 3 is an enlarged cross-section of FIG. 2 taken along line X-X.

FIG. 4 is a cross-section of a flat type disposable diaper incorporating a second embodiment of the present invention, corresponding to FIG. 3.

FIG. 5 is a cross-section of a flat type disposable diaper incorporating a third embodiment of the present invention, corresponding to FIG. 3.

FIG. 6 is a cross-section of a flat type disposable diaper incorporating a fourth embodiment of the present invention, corresponding to FIG. 3.

### DETAILED DESCRIPTION OF THE INVENTION

The disposable diaper of U.S. Pat. No. 4,685,915A provides poor protection against side leakage due to absence of a pair of anti-leakage cuffs elasticized with a standing gather-forming elastic member. If a pair of elasticized anti-eakage cuffs are provided along the longer sides of the absorbent member of the diaper disclosed, another problem can occur as follows.

The disposable diaper of U.S. Pat. No. 4,685,915A is thinner and less stiff in its rear section than in the rest of it because the absorbent member has a less weight per unit area in its rear portion than in its longitudinally middle and front portions. If a pair of elasticized leg flaps and a pair of elasticized anti-leakage cuffs are provided along both lateral sides of the absorbent member in the rear section of the diaper, the rear section would be gathered in the lateral direction in its relaxed state. The gathering in the rear section will make it difficult to flatten the rear section in diapering, that is, a diaperer will have difficulty in applying the rear section of the diaper to the back of a wearer.

When the rear section is designed with a pattern on its garment facing side, the patterned area will also be gathered to impair the visibility of the pattern.

Although the flat type disposable diaper of US 2003236510A1 does not have gather-forming elastic members contractible in the diaper longitudinal direction, the same inconveniences as described above would occur if a pair of elasticized anti-leakage cuffs and a pair of elasticized leg flaps are provided along both lateral sides of the diaper.

The present invention relates to a flat type disposable diaper having standing gathers and leg gathers and yet capable of being flattened in its rear section and thereby easy to apply to the back of a wearer.

The present invention provides a flat type disposable diaper having a topsheet, a backsheet, an absorbent member interposed between the topsheet and the backsheet, a pair of anti-leakage cuffs elasticized with a standing gather-forming elastic member, and a pair of leg flaps elasticized with a leg gather-forming elastic member. The diaper has a longitudinal direction and is sectioned into a front section, a crotch section, and a rear section along the longitudinal direction. The absorbent member includes a higher weight region and a lower weight region. The lower weight region is located in the rear section, and the higher weight region in the crotch section. At least one of the standing gather and the leg gather is substantially absent in the rear section where the higher weight region of the absorbent member is provided.

The flat type disposable diaper of the present invention has standing gathers and leg gathers and is yet easily flattened in its rear section and therefore easy to apply to a wearer's body.

The flat type disposable diaper of the invention will be described generally based on its preferred embodiments with reference to the accompanying drawings.

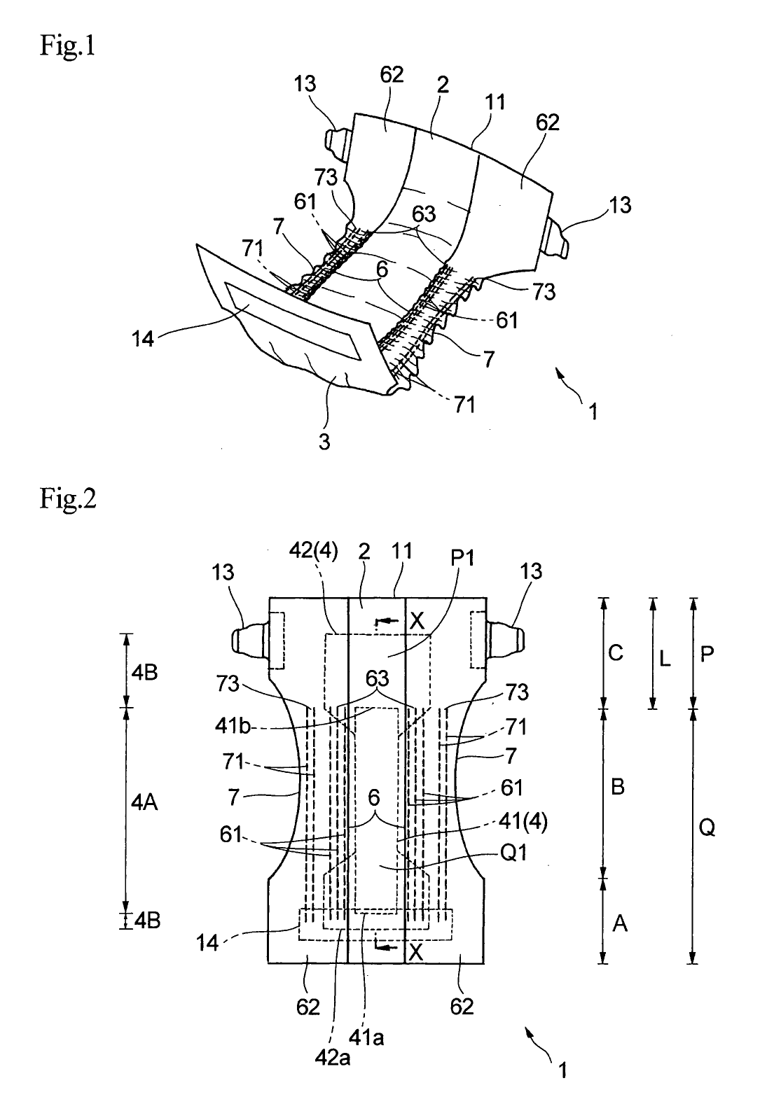

FIGS. 1 through 3 illustrate a flat type disposable diaper 1 incorporating a first embodiment of the invention, which is designed for use by infants. The diaper 1 has a liquid permeable topsheet 2, a liquid impermeable or repellent backsheet 3, and a liquid retentive absorbent member 4 interposed between the topsheet 2 and the backsheet 3. It is substantially oblong with a longitudinal direction and a lateral direction, The diaper 1 has, along the longitudinal direction thereof; a pair of anti-leakage cuffs 6 elasticized with elastic members 61 for the formation of standing gathers and a pair of leg flaps 7 elasticized with elastic members 71 for the formation of leg gathers. The diaper 1 is sectioned in the longitudinal direction into a front section A applied to the front side of a wearer, a crotch section B applied to the crotch of a wearer, and a rear section C applied to the back side of a wearer.

The absorbent member 4 has a higher weight region 4A and a lower weight region 4B. The lower weight region 4B is located in the rear section C, and the higher weight region 4A is in the crotch section. There is no substantial standing gather nor substantial leg gather in the rear section C where the lower weight region 4B is provided.

The diaper 1 of the first embodiment has the shape of a sandglass in its plan with its longitudinally middle portion narrowed. The topsheet 2 and the backsheet 3 each extend laterally outward from both side edges of the absorbent member 4. The backsheet 3 delineates the outline of the diaper 1. The topsheet 2 has a smaller width (in the lateral direction of the diaper 1) than the backsheet 3, and both side edges of the topsheet 2 are inward of both side edges of the backsheet 3. The backsheet 3 has an exterior sheet of water repellent nonwoven fabric joined to the garment facing side thereof The exterior sheet has the same shape as the backsheet 3.

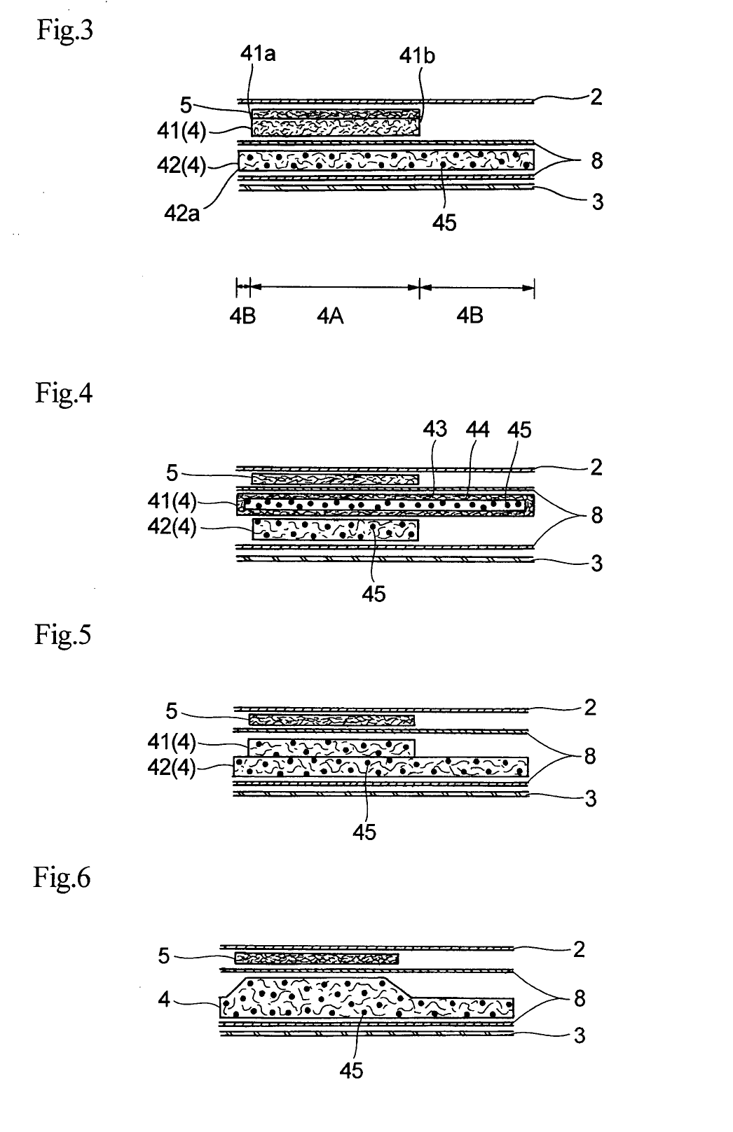

As illustrated in FIG. 3, the absorbent member 4 has a laminate structure composed of an upper absorbent submember 41 and a lower absorbent submember 42 different in length. The overlapping portion between the upper and lower absorbent submembers provides a higher weight region 4A while the unoverlapping portion provides a lower weight region 4B. The upper absorbent submember 41 is shorter than the lower absorbent submember 42.

As illustrated in FIG. 2, the lower absorbent submember 42 is vertically long with its longitudinal direction coinciding with the diaper longitudinal direction and has a front end 42a and an opposite rear end. The lower absorbent submember 42 is disposed from near one of the longitudinal ends of the diaper to near the other (from near the front end of the front section A to near the rear end of the rear section C). The lower absorbent submember 42 has the shape of a sandglass, i.e., with its longitudinally middle portion narrowed in the plan view. The lower absorbent submember 42 is disposed so that the narrowed portion thereof is positioned generally on the crotch section B to give better comfort to the wearer's crotch. The lower absorbent submember 42 is wrapped in paper 8.

As illustrated in FIG. 2, the upper absorbent submember 41 is rectangular with its longitudinal direction coinciding with the diaper longitudinal direction and has a front end 41a and an opposite rear end 41b. The width of the upper absorbent submember 41 is practically the same as the longitudinally middle, narrowed portion of the lower absorbent submember 42. The upper absorbent submember 41 extends from the front section A toward the rear section C.

As shown in FIG. 2, the upper absorbent submember 41 is disposed with its front end 41a slightly longitudinally inward of the front end 42a of the lower absorbent submember 42.

Accordingly, the absorbent member 4 has the higher weight region 4A from the front section A to the crotch section B to exhibit sufficient absorbing capacity for receiving a large amount of urine from an infant wearer.

The diaper 1 has two longitudinally opposite ends, one of which is a rear end 11 in the rear section C, the other is a front end in the front section A. The lower weight region 4B which is located in the rear section C has a rear end nearer to the rear end 11 of the diaper 1 and an opposite front end farther from the rear end 11. The distance L (see FIG. 2) from the rear end 11 of the diaper 1 to the farthest position of the lower weight region 48 in the rear section C from the rear end 11 as measured in the diaper longitudinal direction is preferably 25% to 35%, more preferably 30% to 35%, of the length of the diaper 1 in its flat-out state. In the present embodiment, the farthest position of the lower weight region 4B in the rear section C is equal to the position of the rear end 41b of the upper absorbent submember 41. The term “flat-out state” of the diaper as used herein means the flattened state of the diaper with all the elastic members 61 and 71 disposed in the anti-leakage cuffs 6 and the leg flaps 7 being stretched out flat.

When the distance L is at least 25% of the stretched out length of the diaper, the rear section C is easy to apply to the back of a wearer. The length L being not longer than 35% of the stretched out length of the diaper is preferred to secure the absorbing performance of the absorbent member 4 in the crotch section B.

The portion of the diaper 1 from the rear end 11 up to the rear end 41b of the upper absorbent submember 41 will hereinafter be referred to as a rear portion P. While the rear portion P of the diaper 1 shown in FIG. 2 is the same as the rear section is C, the rear portion P does not always coincide with the rear section C.

For instance, the rear end 41b of the upper absorbent submember 41 may be positioned in the crotch section B, just in short of the border with the rear section C. In this case, the rear section C having the lower weight region 4B is smaller than the rear portion P. Alternatively the rear end 41b may be positioned in the rear section C beyond the boarder. In this case, the rear portion P is smaller than the rear section C.

The part of the absorbent member 4 existing in the rear portion P is the lower weight region 4B formed solely by the lower absorbent submember 42. In the present embodiment, the absorbent member 4 has another lower weight region 4B, which is formed solely by the lower absorbent submember 42, near the front end of the diaper 1 opposite to the rear end 11 of the diaper 1.

The portion of the diaper except the rear portion P, i.e., the portion adjacent to and forward of the rear portion P will hereinafter be referred to as a crotch-to-front portion Q. The crotch-to-front portion Q has the higher weight region 4A of the absorbent member 4.

The higher weight region 4A of the absorbent member 4 preferably has a weight of 500 to 1200 g/cm², more preferably 600 to 1000 g/cm², to give a wearer comfort and to secure absorbency at the time of large amount of infant's urination The lower weight region 4B, on the other hand, preferably has a weight of 100 to 450 g/cm², more preferably 150 to 400 g/cm², to give a wearer comfort in the rear portion P while securing absorbency.

The regional weight of the absorbent member 4 can be measured, for example, as follows. A square test piece measuring 5.0 cm by 5.0 cm in its plan and in its flat-out state is cut out of a region to be measured. The upper absorbent submember 41 or/and the lower absorbent submember 42 is taken out of the cut piece and weighed, and the weight is converted to a per unit area basis.

The diaper 1 of the first embodiment further has a sublayer 5 between the topsheet 2 and the upper absorbent submember 41 as illustrated in FIG. 3. The sublayer 5 has substantially the same shape as the upper absorbent submember 41 in the plan and joined to the skin facing side of the upper absorbent submember 41.

The sublayer 5 helps liquid penetrate from the topsheet 2 into the absorbent member 4, suppresses wet-back, and improves dryness of the topsheet 2. To perform these functions, the sublayer 5 is preferably formed of a bulky sheet with some thickness. The bulk of the sublayer 5 secures the space through which liquid passes from the topsheet 2 to the absorbent member 4, which will accelerate smooth liquid migration From that viewpoint, the sublayer 5 is preferably formed of a hydrophilic nonwoven fabric or an aggregate of hydrophilic staple fiber and preferably has a thickness of 0.1 to 5 mm, more preferably 0.2 to 3 mm.

As illustrated in FIG. 2, the diaper 1 has a pair of anti-leakage cuffs 6 along opposite longer sides of the absorbent member 4. Each cuff 6 is formed of a side sheet 62 elasticized with elastic members 61 and disposed to straddle each longer side edge of the topsheet 2. Each side sheet 62 is fixed to the topsheet 2 at a linear joint extending in the diaper longitudinal direction between each longer side edge of the absorbent member 4 and leg gather-forming elastic members 71 hereinafter described. The linear joint is the base or fixed end of the anti-leakage cuff 6.

The part of the side sheet 62 outboard of its fixed end is bonded to the topsheet 2 or the backsheet 3, and the part of the side sheet 62 inboard of the fixed end and near the opposite front and rear ends of the diaper is bonded to the topsheet 2. The pair of anti-leakage cuffs 6 are each elasticized with elastic members 61 of thread form that are disposed in their stretched state close to the free edge of each cuff 6. The free edge of each cuff 6 is virtually parallel to the diaper longitudinal direction in the flat-out state

The term “standing gather” as used in the invention indicates a gathered part of the anti-leakage cuff 6 gathered by the contraction of the elastic members 61 in the diaper's relaxed state. A part of the cuff 6 where an elastic member 61 is disposed but not contracts in the diaper's relaxed state is not called “standing gather”. The “relaxed state” of a diaper as referred to in the description is defined as follows. A folded disposable diaper is unfolded, once stretched out flat on a horizontal surface until all the gathered parts are filly smoothed out, and then let free from any outer force except gravity to take on a shape with gathering. That state of the diaper is called “relaxed state”.

The expression “standing gather is substantially absent” or “no substantial standing gather” as used herein means there is no gathering due to intentional contraction of the elastic members 61. If an elastic member 61 is unintentionally bonded to a part of the side sheet 62 in its stretched state during the manufacture of the diaper to make gathering, such a gathered part is not called “standing gather”. Neither are wrinkles nor creases of the side sheet 62 per se called “gather”. The same interpretation applies to the expression “leg gather is substantially absent”.

The diaper 1 has no substantial standing gather in the rear portion P. There is no gather-forming elastic member 61 disposed in the rear portion P or, if any, the elastic member 61 is fixed to the side sheet 62 in its non-stretched state.

Because the lower weight region 4B of the absorbent member 4 in the rear portion P is not affected by the elastic members 61 contracting in the diaper's relaxed state, no wrinkles or folds due to gathering of the absorbent member 4 are created in the rear portion P and on the boundary between the rear portion P and the crotch-to-front portion Q. In the absence of the standing gather in the rear portion P, no marks of gathering will be left on the skin of a wearer having been in contact with the rear portion P.

The standing gather has a rear end 63 nearer to the rear end 11 of the diaper 1 and an opposite front end farther from the rear end 11. The rear end 63 is even with the rear end 41b of the upper absorbent submember 41. Accordingly, the length from the rear end 11 of the diaper 1 to the rear end 63 of the standing gather as measured in the longitudinal direction of the flat-out diaper 1 is equal to the distance L of the rear portion P.

The distance L is measured, for example, as follows. The diaper 1 in its relaxed state is marked at the intersection between the rear end 11 of the diaper 1 and the free edge of the anti-leakage cuff 6 or its extension and at the rear end 63 of the standing gather. The diaper 1 is then stretched out flat, and the distance between the two marks is measured to give the distance L.

The diaper 1 of the first embodiment also has a pair of leg flaps 7 along the opposing longer sides thereof as illustrated in FIG. 2. The leg flaps 7 are formed of the pair of side sheets 62 laterally extending in opposite directions from the fixed end of each anti-leakage cuff 6. The pair of leg flaps 7 are each elasticized with elastic members 71 of thread form that are fixed in their stretched state close to the free edge of each leg flap 7.

The term “leg gather” as used in the invention indicates a gathered part of the leg flap 7 gathered by the contraction of the elastic members 71 in the diaper's relaxed state. A part of the leg flap 7 where an elastic member 71 is disposed but not contracts in the diaper's relaxed state is not called “leg gather”.

When in the relaxed state, the diaper 1 is curved about a transverse axis with the skin facing side inside by the gathering of the elasticized anti-leakage cuffs 6 and of the elasticized leg flaps 7.

The diaper 1 according to the first embodiment has no substantial leg gather in the rear portion P. There is no gather-forming elastic member 71 disposed in the rear portion P or, if any, the elastic member 71 is fixed to the side sheet 62 in its non-stretched state.

Because the lower weight region 4B of the absorbent member 4 in the rear portion P is not affected by the elastic members 71 contracting in the diaper's relaxed state, no wrinkles or folds due to gathering of the absorbent member 4 are created in the rear portion P and on the boundary between the rear portion P and the crotch-to-front portion Q. In the absence of the leg gather in the rear portion P, no marks of leg gathering will be left on the skin of a wearer having been in contact with the rear portion P.

The leg gather has a rear end 73 nearer to the rear end 11 of the diaper 1 and an opposite front end farther from the rear end 11. The rear end 73 is even with the rear end 41b of the upper absorbent submember 41. Accordingly, the length from the rear end 11 of the diaper 1 to the rear end 73 of the leg gather as measured in the longitudinal direction of the flat-out diaper 1 is equal to the distance L of the rear portion P.

It is effective in helping the elasticized anti-leakage cuffs 6 stand upright and is therefore preferred in the first embodiment that the length of the elastic members 61 for the standing gather formation be larger than that of the elastic members 71 for the leg gather formation. The term “length” of an elastic member as referred to herein means the length of the elastic member measured when it is fixed to the diaper 1 in its stretched state and measured in the longitudinal direction of the diaper 1. To ensure the above effect, the ratio of the length L61 of the standing gather-forming elastic members 61 to the length L71 of the leg gather-forming elastic members 71 (i.e., L61/L71) is preferably 1.05 to 1.30, more preferably 1.10 to 1.20.

The diaper 1 incorporating the structure according to the first embodiment features regionally varied unit area weight of the part thereof where the absorbent member 4 is disposed That is, the portion P1 of the rear portion P where the lower weight region 4B is located has a relatively small weight, whereas the portion Q1 of the crotch-to-front portion Q where the higher weight region 4A is located has a relatively large weight.

More specifically, the weight of the portion P1 is preferably 150 to 600 g/cm², more preferably 200 to 550 g/cm². When the portion P1 has a weight of 150 g/cm² or more, capability of absorbing the excrements migrating into the rear portion P is secured. When the portion P1 has a weight of 600 g/cm² or less, improvement of wearing comfort, improvement of packaging efficiency, and cost reduction can be promoted.

The weight of the portion Q1 is preferably 550 to 1350 g/cm², more preferably 650 to 1150 g/cm². The weight of 550 g/cm² or more ensures absorbency at the time of large amount of infant's urination. The weight of 1350 g/cm² or less assures comfort.

The regional weight of the diaper 1 is measured, for example, as follows. A square specimen measuring 5.0 cm by 5.0 cm in its plan and in its flat-out state is cut out of a region to be measured of the diaper. The specimen is weighed, and the weight is converted to a per unit area basis.

The diaper 1 incorporating-the first embodiment also features regionally varied thickness of the portion where the absorbent member 4 is disposed. That is, the is portion P1 of the rear portion P where the lower weight region 4B is located 1 has a relatively small thickness, whereas the portion Q1 of the crotch-to-front portion Q where the higher weight region 4A is located has a relatively large thickness.

More specifically, the thickness of the portion P1 is preferably 2.0 to 5.0 mm, more preferably 1.5 to 4.0 mm, and that of the portion Q1 is preferably 3.5 to 8.0 mm, more preferably 4.0 to 7.0 mm. These preferences are based on the same reasons as described with respect to the weight.

The thickness of the diaper 1 is measured, for example, as follows. A 5.0 cm-square test piece is cut out of each region of the diaper in the flat-out state. Measurement is made with a laser displacement sensor PA-1830 from Keyence Corp. A plate of the same size as the test piece is mounted on the measuring stage. The vertical position of the upper surface of the plate is taken as a base point A. The plate is once removed, the test piece placed on the measuring stage, and the plate put thereon again. The vertical position of the upper surface of the plate is taken as point B. The difference between points A and B is taken as the thickness of the test piece. The weight of the plate is such that applies a pressure of 245 Pa to the test piece. Thickness of the test piece cut out of each region was measured at five arbitral portions, and its average value is to be the thickness of the region of the diaper 1.

The diaper 1 of the first embodiment also features regionally varied bending stiffness of the portion where the absorbent member 4 is disposed in the diaper longitudinal direction. That is, the portion P1 of the rear portion P where the lower weight region 4B has a relatively low bending stiffness, while the portion Q1 of the crotch-to-front portion Q where the higher weight region 4A is located has a relatively high bending stiffness.

More specifically, the portion P1 preferably has a bending stiffness of 35 cN or lower, more preferably 25 cN or lower, to give better comfort to a wearer and to facilitate applying the diaper 1 to a wearer.

The bending stiffness of the portion Q1, on the other hand, is preferably 40 to 120 cN, more preferably 50 to 100 cN. With this bending stiffness being 40 cN or higher, the portion Q1 in the front section A is prevented from writing or bunching by the influences of the standing gathers and leg gathers and therefore secures ease of diapering. The bending stiffness of 120 cN or lower of the portion Q1 assures infant's comfort.

The regional bending stiffness of the diaper 1 is measured, for example, as follows. A handle-o-meter specified in JIS L-1096 (Model HOM-2) can be used for the measurement A 5.0 cm-side square specimen is cut out of the portion P1 or Q1 of the diaper 1 in its flat-out state. The specimen is placed on the platform having a 30 mm wide slot with the diaper's longitudinal direction perpendicular to the slot. The resistance (g) encountered by the 2 mm thick penetrator blade as it is moved into the slot by 8 mm is measured. The measurement is taken on three points to obtain an average.

As described, although the portion of the rear portion P where the absorbent member 4 is disposed has a relatively small weight, small thickness and low bending stiffness, the rear portion P is free from wrinkles or folds and is easily flattened when put on a wearer because there is neither standing gather nor leg gather in the rear portion P in relaxedl state of diaper.

The structure and material of the diaper 1 of the first embodiment will further be described.

The diaper 1 has a pair of fastening tapes 13 provided on each lateral side of the rear section C to stick out laterally. A landing zone 14 adapted to receive the fastening tapes 13 is provided on the garment facing side of the front section K

Each fastening tape 13 includes a base and a male member of a mechanical fastener fixed on one side of the base. The fastening tape 13 has a fixed part (the invisible part indicated by the dotted line adjacent to the visible part of the fastening tape 13 in FIG. 2) and a sticking part extending laterally outward from the side edge of the rear section C. The fixed part is fixed between the topsheet 2 and the backsheet 3. The fixed part is formed of the base only, and the sticking part is composed of the base and the male member attached thereto. Any male member known in the art can be used.

The base of the fastening tape 13 is preferably made of an extensible and contractible sheet material Fastening with fastening tape 13 having an extensible and contractible base achieves improved fit and prevents diaper drooping without using diaper wrinkling Examples of the extensible and contractible sheet materials include films of thermoplastic elastomer such as urethane elastomer and styrene elastomer, nonwoven fabrics made of fibers of such thermoplastic elastomer, and extensible and contractible composite fabrics composed of a sheet at least a part of which is made of an extensible and contractible elastomer and fabric having extensibility (including woven, knits, and nonwovens) joined to at least one side of the sheet. The extensible and contractible composite fabric composed of a sheet at least a part of which is made of an extensible and contractible elastomer and fabric having extensibility joined to at least one side of the sheet is particularly preferred.

The extensible and contractible composite fabric preferably has a permanent set of 30% or less and a load at 100% elongation of 100 g/25 mm or more. Fasting tapes having, as a base, the extensible and contractible composite fabric having a permanent set and a load at 100% elongation fig within the respective recited ranges are deformable in conformity the movement of a wearer without being destroyed. The permanent set as referred to above is measured as follows A specimen measuring 25 mm in width and 150 mm in length is set on a tensile tester at an initial gauge length of 100 mm. The specimen is extended 100% at a pulling speed of 300 mm/min and then released from the tension. The length of the specimen after the release (length after extension) is measured. The permanent set is calculated from equation: Permanent set (%)=[(length after extension−initial gauge length)/initial gauge length]×100

The landing zone 14 is formed of a base and a female member of a mechanical fastener bonded to the base. The base is made of nonwoven fabric, resin film, etc. Any material that the male member can engage on being pressed can be used as the female member.

Materials from which the upper absorbent submember 41 can be configured include various types of pulp commonly used in conventional absorbent articles. Softwood pulp, chemically treated synthetic pulp, and continuous fiber such as a cellulose tow are preferred. Crosslinked pulp with a highly bulky (twisted) structure is particularly preferred. An absorbent submember made of such crosslinked pulp retains bulkiness not only while dry but while wet after absorption.

Materials from which the lower absorbent submember 42 include those recited above as materials of the upper absorbent. Submember 41 and, in addition, superabsorbent polymer 45 (see FIG. 3), synthetic fibers such as polyester fiber and polypropylene fiber, heat fusible fiber, and rayon. Mixtures of the pulp with other materials recited are also preferred A mixed aggregate of the pulp and the superabsorbent polymer 45 is particularly preferred.

Examples of the superabsorbent polymer 45 include sodium polyacrylate, acrylic acid-vinyl alcohol copolymers, crosslinked sodium polyacrylate, starch-acrylic acid graft polymers, isobutylene-maleic anhydride copolymers and saponification products thereof, potassium polyacrylate, and cesium polyacrylate.

The topsheet 2, the backsheet 3, the sublayer 5, the wrapping paper 8, and the exterior sheet can be made from various respective materials commonly employed in the art.

The diaper 1 of the first embodiment produces the following effects.

Since there are no wrinkles or folds nor gathers stretchable in the longitudinal direction in the rear portion P of the diaper 1 in the relaxed state, the skin facing side of the rear portion P comes into close contact with the skin of the wearer' back without forming any gap therebetween.

The part of the absorbent member 4 located in the rear portion P has a reduced weight and thickness, which leads to improved wearing comfort and reduced material cost.

Having a reduced thickness in the rear portion P, the diaper 1 can be folded compact. This is advantageous to increase efficiency in packaging a plurality of diapers 1 as one unit. When taken out of the package, the diaper 1 is free from wrinkles on its rear portion P, which assures visibility of a pattern, if any provided, on the garment facing side of the rear portion P.

In the case when the standing gather-forming elastic members 61 are longer than the leg gather-forming elastic members 71, the elasticized anti-leakage cuffs 6 stand upright more easily and provide a better fit and enhanced leakage protection.

When the base of the fastening tapes 13 is an extensible sheet, the diaper 1 can be fastened without creating wrinkles to provide a better fit and to be prevented from drooping.

Flat type disposable diapers incorporating a second to fourth embodiment of the present invention will then be described with reference to FIGS. 4 through 6. The description on the first embodiment applies to the embodiments described hereunder unless otherwise specified. Elements or members common to both FIGS. 1 to 3 and FIGS. 4 to 6 are given the same numerical and/or alphabetical designation and will not be redundantly described.

The diaper incorporating the second preferred embodiment has an upper absorbent submember 41 which is longer than a lower absorbent submember 42 as illustrated in FIG. 4. The upper absorbent submember 41 has the same shape as the lower absorbent submember used in the first embodiment, and the lower absorbent submember 42 has the same shape as the upper absorbent submember used in the first embodiment.

The overlapping portion of the upper absorbent submember 41 and the lower absorbent submember 42 in the absorbent member 4 provides a higher weight region 4A while the unoverlapping portion provides a lower weight region 4B, which is the same as in the first embodiment.

In the second embodiment, the absorbent member 4, composed of the upper absorbent submember 41 and the lower absorbent submember 42, is wrapped in paper 8 as a unit. A sublayer 5 is interposed between the topsheet 2 and the upper absorbent submember 41 and adjacent to the upper absorbent submember 41 via the wrapping paper 8.

The upper absorbent submember 41 contains a web 44 of hydrophilic continuous fibers 43 and a superabsorbent polymer 45 embedded held in the web 44. The continuous fibers 43 are crimped fibers and generally oriented in the longitudinal direction of the absorbent member 4.

The upper absorbent submember 41 is flexible and stretchable and therefore easily conformingly deformed to the wearer's movement, helping the diaper feel soft to the wearer around the groins and thighs and give great comfort.

It is preferred for the continuous fibers 43 in the area where the superabsorbent polymer 45 is present to have a percentage of crimp (JIS L0208) of 10% to 90%, more preferably 10% to 60%, even more preferably 20% to 50%.

Making the web 44 out of crimped continuous fibers 43 helps embedded hold a large amount of the superabsorbent polymer 45 in the web 44 stably. In the case when the web 44 is made solely of non-crimped or less-crimped continuous fibers, and a large quantity of the superabsorbent polymer 45 is used, the polymer particles tend to extremely move in or fall off the web 44. Conversely, using too much crimped continuous fibers makes it difficult to incorporate the superabsorbent polymer 45 between the continuous fibers, which can also result in extreme movement, or fall-off, of the polymer particles within or out of the web 44. Means for crimping the continuous fibers 43 is not particularly restricted The crimp may be two-dimensional or three-dimensional. The percentage of crimp (or crimp percentage) is defined to be a percentage of a difference between the length A of a continuous fiber 43 in its straightened state and the natural length B of the continuous fiber 43 to the length A, being calculated from equation:
Percentage of crimp (%)=(A−B)/A×100

The natural length of a continuous fiber 43 is the length of the straight line connecting the two ends of a continuous fiber 43 in its natural state. The term “natural state” means a state of a continuous fiber 43 hanging under its own weight with its one end fixed to a horizontal plate. The term “straightened state” means a state of a continuous fiber 43 stretched out until no crimp remains under a minimum load.

The number of crimps of the crimped continuous fibers 43 having the recited percentage of crimp is preferably 2 to 25, more preferably 4 to 20, even more preferably 10 to 20, per centimeter.

Using crimped continuous fibers 43 having a lower crimp percentage within the recited preferred range leads to reduction of the amount of the fibers 43 required to define the longitudinal length of the upper absorbent submember 41, resulting in a smaller thickness of the upper absorbent submember 41.

From the same viewpoint, the weight of the web 44 used in the upper absorbent submember 41 preferably ranges from 10 to 200 g/m², more preferably 20 to 100 g/m².

The continuous fibers 43 in the area of the upper absorbent submember 41 where the superabsorbent polymer 45 is present are generally oriented in the longitudinal direction of the upper absorbent submember 41. Urine, on being introduced into the upper absorbent submember 41 through the topsheet 2, spreads preferentially in the orientation direction of the continuous fibers 43 and is absorbed and retained over practically the whole longitudinal length of the upper absorbent submember 41.

The lower absorbent submember 42 is preferably made of the same materials as used to form the lower absorbent submember 42 of the first embodiment.

According to the configuration of the second embodiment, the diaper produces the same effects as by the first embodiment and, in addition, ensures further improved comfort and absorptivity owing to the above-described upper absorbent submember 41.

In the third preferred embodiment of the invention, the absorbent member 4, composed of the upper absorbent submember 41 and the lower absorbent submember 42, is wrapped in paper 8 as a unit as illustrated in FIG. 5. A sublayer 5 is interposed between the topsheet 2 and the upper absorbent submember 41 and adjacent to the upper absorbent submember 41 via the wrapping paper 8. The diaper of the third embodiment is otherwise structurally the same as the diaper 1 of the first embodiment.

The upper absorbent submember 41 and the lower absorbent submember 42 used in the third embodiment are-preferably made of the same materials recited for use in the first embodiment.

The diaper of the third embodiment produces the same effects as by the first embodiment.

In the diaper incorporating the fourth preferred embodiment of the invention, the upper absorbent submember 41 and the lower absorbent submember 42 constitute a unitary absorbent member 4, which is wrapped in paper 8. A sublayer 5 is interposed between the topsheet 2 and the absorbent member 4 and adjacent to the absorbent member 4 via the paper 8. The diaper of the fourth embodiment is otherwise structurally the same as the diaper 1 of the first embodiment.

The absorbent member 4 is preferably made out of the same materials as used to make the lower absorbent submember used in the first embodiment.

The diaper of the fourth embodiment produces the same effects as by the diaper 1 of the first embodiment.

The flat type disposable diaper of the present invention is not limited to the foregoing embodiments, and various changes and modifications can be added thereto without departing from the spirit and scope of the present invention. For example, the diaper of the invention may have either one of the standing gather and the leg gather in the rear section C having the lower weight region 4B.

The diaper of the invention may have an elasticized part extensible and contractible in the diaper width direction in the rear section C. For example, an elasticized waist part extensible and contractible in the diaper width direction may be provided along the rear end 11 of the diaper, or a pair of elasticized parts extensible and contractible in the diaper width direction may be provided along each lateral side edge of the rear section C.

The flat type disposable diaper of the present invention is designed for use by not only infants but also adults.

In the description given above, particulars of a certain embodiment that have been omitted to avoid redundancy can appropriately be complemented by the corresponding description of other embodiments. Particulars described as a characteristic of a certain embodiment can apply to other embodiments as appropriate.

The present invention will now be illustrated in greater detail by way of Examples, but it should be understood that the invention is not construed as being limited thereto.

### EXAMPLE 1

A flat type disposable diaper illustrated in FIGS. 1 to 3 was made.

The measures were as follows: the distance L of the rear portion P, 31% of the length of the diaper in the flat-out state; the weight, thickness, and bending stiffness of the portion P1 of the rear portion P where the lower weight region 4B was located, 490 g/m², 3.7 mm, and 21 cN, respectively; the weight, thickness, and bending stiffness of the portion Q1 of the crotch-to-front portion Q where the higher weight region 4A was located, 800 g/m², 6.3 mm, and 76 cN, respectively. A pattern was printed on the garment facing side of the rear section C. The size of the pattern was about 35% of the area of the rear section C in its plan view. The whole pattern was within the rear section C.

### EXAMPLE 2

A diaper was made in the same manner as in Example 1, except that the standing gather was formed to extend into the rear portion P. The distance from the rear end 11 of the diaper to the rear end 63 of the standing gather in the rear section C was 18% of the length of the diaper in the flat-out state.

### EXAMPLE 3

A diaper was made in the same manner as in Example 1, except that the leg gather was formed to extend into the rear portion P. The distance from the rear end 11 of the diaper to the rear end 73 of the leg gather in the rear section C was 20% of the length of the diaper in the flat-out state.

### COMPARATIVE EXAMPLE

A comparative diaper was made in the same manner as in Example 1, except that both the standing gather and the leg gather were formed to extend into the rear portion P. The distance from the rear end 11 of the diaper to the rear end 63 of the standing gather in the rear section C was 18% of the length of the diaper in the flat-out state. The distance from the rear end 11 of the diaper to the rear end 73 of the leg gather in the rear section C was 20% of the length of the diaper in the flat-out state.

The diapers of Examples 1 to 3 and Comparative Example were evaluated for ease of flattening in diapering and visibility of the printed pattern. The results of is evaluation are shown in Table 1 below.

(1) Ease of Flattening

The diaper was placed on a horizontal plane in its relaxed state. The state of the rear portion P of the diaper was observed with the naked eye and rated on the following scale.
Very good: The rear portion P was flat with no wrinkles in its relaxed state.
Good: The rear portion P was generally flat but with wrinkles in part.
Bad: The rear portion P had deep wrinkles that could not be smoothed out flat.
   (2) Pattern Visibility

The pattern printed on the rear section C was observed with the naked eye while the diaper was folded along its transverse axis with the topsheet 2 side inside. The visibility of the pattern was rated on the following scale.
Very good: The rear portion P had no wrinkles. The pattern was visible over all.
Good: Although the rear portion P had wrinkles in part, almost the whole pattern was visible.
Moderate: Although the rear portion P had wrinkles to hide part of the pattern, the pattern is still recognizable as a whole.

Bad: The rear portion P had deep wrinkles to hide part of the pattern, making the pattern unpresentable and unrecognizable as a whole.

TABLE 1

| | | | | | Compara. |
|---|---|---|---|---|---|
| | | Example 1 | Example 2 | Example 3 | Example |
| Configur- | Standing | no | yes | no | yes |
| ation | Gather | | | | |
| in Rear | Leg Gather | no | no | yes | yes |
| Portion P | | | | | |

| | | | | |
|---|---|---|---|---|
| Ease of Flattening | very good | good | good | bad |
| Pattern Visibility | very good | good | moderate | bad |

As is apparent from the results in Table 1, the diapers of Examples 1 to 3 are superior to the comparative one in ease of flattening in diapering and visibility of the printed pattern.

What is claimed is:

1. A flat type disposable diaper having a longitudinal direction and a lateral direction and sectioned into a front section, a crotch section, and a rear section having a rear end along the longitudinal direction, the diaper comprising a topsheet, a backsheet, an absorbent member interposed between the topsheet and the backsheet, a pair of anti-leakage cuffs elasticized with a standing gather-forming elastic member, and a pair of leg flaps elasticized with a leg gather-forming elastic member,
the absorbent member including a higher weight region and a lower weight region,
the lower weight region being in the rear section,
the higher weight region being in the crotch section,
at least one of the standing gather and the leg gather is substantially absent in the rear section where the lower weight region of the absorbent member is disposed.

2. The flat type disposable diaper according to claim 1, wherein both the standing gather and the leg gather are substantially absent in the rear section where the lower weight region of the absorbent member is disposed.

3. The flat type disposable diaper according to claim 1, wherein the distance from the rear end of the diaper to the farthest position of the lower weight region in the rear section from the rear end of the diaper as measured in the diaper longitudinal direction is 25% to 35% of the length of the diaper in its flat-out state.

4. The flat type disposable diaper according to claim 1, wherein the absorbent member comprises a laminate of an upper absorbent submember and a lower absorbent submember different in length, the higher weight region is the overlapping portion between the submembers, and the lower weight region is the unoverlapping portion between the submembers.

5. The flat type disposable diaper according to claim 1, wherein the portion of the diaper where the lower weight region is located has a weight of 100 to 600 g/cm².

6. The flat type disposable diaper according to claim 1, further comprising a pair of fastening tapes provided on each lateral side of the rear section to stick out laterally, the fastening tapes each comprising an extensible sheet as a base.

7. The flat type disposable diaper according to claim 1, wherein the standing gather-forming elastic member is longer than the leg gather-forming elastic member.
